(19) United States
(12) Patent Application Publication
(10) Pub. No.: US 2006/0122160 A1
(43) Pub. Date: Jun. 8, 2006

(54) Co-administration of dehydroepiandrosterone (DHEA) congener with parthenolide for treating inflammation

(76) Inventors: Dinesh Patel, Salt Lake City, UT (US); David Vollmer, West Jordan, UT (US); Claire Daugherty, Salt Lake City, UT (US); John Gebhard, Salt Lake City, UT (US)

Correspondence Address: THORPE NORTH & WESTERN, LLP., 8180 SOUTH 700 EAST, SUITE 200, SANDY, UT, 84070, US

(21) Appl. No.: 11/270,326

(22) Filed: Nov. 8, 2005

Related U.S. Application Data
(63) Continuation-in-part of application No. 11/145,024, filed on Jun. 3, 2005.
(60) Provisional application No. 60/584,350, filed on Jun. 30, 2004.

(51) Int. Cl. A61K 31/57; A61K 31/365

(52) U.S. Cl. 514/171; 514/468

(57) ABSTRACT

The present invention is related to therapeutic uses of dehydroepiandrosterone (DHEA) congeners. More specifically, the present invention relates to the co-administration of a dehydroepiandrosterone (DHEA) congener in combination with a parthenolide to reduce inflammation.

### PRIORITY DATA

The present application is a continuation-in-part of U.S. patent application Ser. No. 11/145,024, filed Jun. 3, 2005, which claims the benefit of U.S. Provisional Application No. 60/584,350 filed Jun. 30, 2004, both of which are incorporated herein by reference in their entireties.

### FIELD OF THE INVENTION

The present invention is drawn to methods of reducing inflammation. More particularly, the present invention relates to the co-administration of a dehydroepiandrosterone (DHEA) congener in combination with a parthenolide to reduce inflammation.

### BACKGROUND OF THE INVENTION

Inflammation within a human subject is a common physiological response by the immune system to an injury or irritation, where the irritation can be by infectious, allergic, and/or chemical irritants. Some of the clinically observable symptoms of inflammation include increased redness, temperature, swelling, and pain, as well as the loss of function within the inflamed area. These symptoms can be a direct result of infiltration of body fluids and leucocytes (white blood cells) into the inflamed area. This physiological response can be beneficial for the subject because of the ability of the body fluids to dilute any present toxins or substances, to facilitate the entry of antibodies, nutrients, oxygen, and immunological cells to the site, and to aid in drainage from the site. Additionally, leucocytes can aid in destroying any foreign substance within the inflamed area.

While inflammation can primarily be a favorable defense mechanism, it can also have unfavorable consequences when it is an inappropriate immunological response incited by a non-harmful substance. Additionally, diseases such as human inflammatory disorders, infectious disorders, and autoimmune disorders can also result in unfavorable inflammation.

An abbreviated listing of some inflammatory diseases includes arthritis, bronchitis, allergic rhinitis, atopic dermatitis, chronic cholecystitis. Additionally, inflammation arising from a disease or an inappropriate immunological response can have other physiological effects that may not be desirable including fever, malaise, nausea, enlarged lymph nodes, increased erythrocyte sedimentation, and leucocytosis. As such, most subjects do not want to suffer from any diseases or inappropriate immunological responses that result in inflammation, and desire treatment and/or prevention of such inflammation and associated maladies. Thus, research and development continues to seek pharmaceutical products for reducing inflammation.

### SUMMARY OF THE INVENTION

It has been recognized that it would be advantageous to provide methods for reducing inflammation in a subject, and for treating other associated maladies. As such, the present invention provides for methods of reducing inflammation in a subject. One of these methods can include co-administering a therapeutically effective amount of a DHEA congener and a parthenolide to the subject. In a particular aspect of this method, a DHEA congener and parthenolide may be co-administered to the subject. Compositions suitable for carrying out these methods are also provided.

Additional features and advantages of the invention will be apparent from the detailed description, which illustrates, by way of example, features of the invention.

### BRIEF DESCRIPTION OF THE DRAWINGS

FIG. 1 is a graphical representation depicting the effect of differing amounts of DHEA and parthenolide alone, as well as in combination, on in vitro TNF-α production by THP-1 macrophages.

### DETAILED DESCRIPTION OF THE INVENTION

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

The singular forms “a,” “an,” and “the” include plural referents unless the context clearly dictates otherwise. Thus, for example, reference to “a drug” includes reference to one or more of such drugs.

As used herein, the terms “formulation” and “composition” may be used interchangeably and refer to a combination of a pharmaceutically active agents, such as a DHEA congener formulated with one or more additional anti-inflammatory agent(s). The terms “drug,” “active agent,” “bioactive agent,” “pharmaceutically active agent,” and “pharmaceutical,” can also be used interchangeably to refer to an agent or substance that has measurable specified or selected physiologic activity when administered to a subject in an effective amount. These terms of art are well known in the pharmaceutical and medicinal arts.

As used herein, “administration,” and “administering” refer to the manner in which a drug, formulation, or composition is introduced into the body of a subject. Administration can be accomplished by various art-known routes such as oral, parenteral, transdermal, inhalation, implantation, etc. Thus, an oral administration can be achieved by swallowing, chewing, or sucking an oral dosage form comprising active agent(s). Parenteral administration can be achieved by injecting a drug composition intravenously, intra-arterially, intramuscularly, intrathecally, or subcutaneously, etc. Transdermal administration can be accomplished by applying, pasting, rolling, attaching, pouring, pressing, rubbing, etc., of a transdermal preparation onto a skin surface. Nasal administration may be achieved by inhaling a vapor, mist, or aerosol of the a preparation comprising the active agent(s), or by other techniques that allow the preparation to enter the respiratory system via the nasal passages or that permit the preparation to be absorbed by the nasal mucosa. These and additional methods of administration are well known in the art.

The term “co-administering,” co-administration,” or “co-administer” refers to the administration of a DHEA congener with another anti-inflammatory agent such as parthenolide. Both the DHEA congener and the second anti-inflammatory agent can be administered simultaneously, or at different times, as long as these active agents work in concert to produce a physiological effect. Additionally, co-administration does not require the DHEA congener and the second anti-inflammatory agent to be administered by the same route. As such, each can be administered independently or as a common dosage form.

The terms “effective amount,” and “sufficient amount” may be used interchangeably and refer to an amount of an ingredient which, when included in a composition, is sufficient to achieve an intended compositional or physiological effect. Thus, a “therapeutically effective amount” refers to a non-toxic, but sufficient amount of an active agent, to achieve therapeutic results in treating a condition for which the active agent is known to be effective. Various biological factors may affect the ability of a substance to perform its intended task. Therefore, an “effective amount” or a “therapeutically effective amount” may be dependent on such biological factors. Further, while the achievement of therapeutic effects may be measured by a physician or other qualified medical personnel using evaluations known in the art, it is recognized that individual variation and response to treatments may make the achievement of therapeutic effects a subjective decision. In some instances, a “therapeutically effective amount” of a drug can achieve a therapeutic effect that is measurable by the subject receiving the drug. The determination of an effective amount is well within the ordinary skill in the art of pharmaceutical, medicinal, and health sciences. See, for example, Meiner and Tonascia, “Clinical Trials: Design, Conduct, and Analysis,” Monographs in Epidemiology and Biostatistics, Vol. 8 (1986), which is incorporated herein by reference.

As used herein, the terms “inhibit” or “inhibiting” refers to the process of holding back, suppressing or restraining so as to block, prevent, limit, or decrease a rate of action or function. The use of the term is not to be misconstrued to be only of absolute prevention, but can be a referent of from any incremental step of limiting or reducing a function to the full and absolute prevention of the function. In one example, when the term “inhibit” is utilized in combination with a substance, such as an immune mediator responsive to TNF-α, inhibition of the production of the substance can include the reduction of the production and/or secretion of the substance. Alternatively, TNF-α receptors can be inhibited from receiving TNF-α.

As used herein, “reduce” or “reducing” refers to the process of decreasing, diminishing, or lessening, as in extent, amount, or degree of that which is reduced. The use of the term with respect to inflammation can include any incremental step that results in less inflammation, such as less redness, temperature, swelling, and/or pain. Additionally, the use of the term can include from any minimal decrease to absolute abolishment of a physiological process or effect.

As used herein, “treat,” “treatment,” or “treating” refers to the process or result of giving medical aid to a subject, where the medical aid can counteract a malady, a symptom thereof, or other related adverse physiological manifestation. Additionally, these terms can refer to the administration or application of remedies to a patient or for a disease or injury; such as a medicine or a therapy. Accordingly, the substance or remedy so applied, such as the process of providing procedures or applications, are intended to relieve illness, injury or inflammation. Additionally, the term can be used for the procedure of preemptively acting to prevent the malady, a symptom thereof, or other related adverse physiological manifestation. As such, a treatment can be administered prior to the subject experiencing any symptoms so that the symptoms are not manifested in the subject.

As used herein, “carrier” or “inert carrier” refers to typical compounds or compositions used to carry active ingredients, such as polymeric carriers, liquid carriers, or other carrier vehicles with which a bioactive agent, such as a DHEA congener and/or other anti-inflammatory agents, may be combined to achieve a specific dosage form. As a general principle, carriers do not substantially react with the bioactive agent in a manner which substantially degrades or otherwise adversely affects the bioactive agent or its therapeutic potential.

As used herein, “subject” refers to an animal, such as a mammal, that may benefit from the administration of an inflammation reducing drug, a combination of drugs, or a formulation; or from a method for achieving reduced inflammation recited herein. Most often, the subject will be a human.

The term “dehydroepiandrosterone congener” or “DHEA congener” includes dehydroepiandrosterone (a.k.a. DHEA and (3β)-3-hydroxyandrost-5-en-17-one), derivatives of DHEA, metabolites of DHEA, metabolites of DHEA derivatives, salts of DHEA, salts of DHEA derivatives, etc. DHEA, generally, is a weak androgen that serves as the primary precursor in the biosynthesis of both androgens and estrogens. Typically, a DHEA congener used in accordance with embodiments of the present invention is in a pharmaceutically acceptable form.

As used herein, “mc” or “micro” when used in combination with a unit of measurement denotes the standard unit to be divided by one million, or multiplied by 1×10⁻⁶. Accordingly, the prefix “micro,” which is well known by one or ordinary skill in the art can be referred herein by the abbreviation “mc.”

As used herein, “mg/kg” or any other mass unit divided by another mass unit when used to describe a drug dose or dosing regimen denotes the mass of drug delivered per mass of the subject being administered the drug. Such use of units when referring to pharmaceuticals and their associated doses is well known to one of ordinary skill in the art.

As used herein, “mg/m²” or any other mass unit divided by an area unit when used to describe a drug dose or dosing regimen denotes the mass of the drug delivered per surface area of the subject being administered the drug. The use of mass of drug per surface area of subject when referring to pharmaceuticals and their associated doses is well known to one of ordinary skill in the art.

As used herein, “enhance” or “enhancing” of an anti-inflammatory response refers to the interaction of two or more active agents or drugs so that their combined physiological effect is greater than the individual effect of either active agent when administered alone at the same dosage. “Synergism” or “synergistic effect” refers to an anti-inflammatory response where the interaction of two or more active agents or drugs provides a combined physiological effect that is greater than the additive effect of both active agents.

The term “anti-TNF-α agent” refers to compositions or compounds that act to inhibit the normal function tumor necrosis factor alpha (TNF-α), which are important cytokines involved in systemic inflammation and the acute phase response.

The term “about” when referring to a numerical value or range is intended to encompass the values resulting from experimental error that can occur when taking measurements.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight range of about 1 wt % to about 20 wt % should be interpreted to include not only the explicitly recited concentration limits of 1 wt % to about 20 wt %, but also to include individual concentrations such as 2 wt %, 3 wt %, 4 wt %, and sub-ranges such as 5 wt % to 15 wt %, 10 wt % to 20 wt %, etc.

A) Dehydroepiandrosterone Congeners

As stated, a DHEA congener includes DHEA (3β)-3-hydroxyandrost-5-en-17-one), derivatives of DHEA, metabolites of DHEA, metabolites of DHEA derivatives, salts of DHEA, salts of DHEA derivatives, etc. Typically, a DHEA congener used in accordance with embodiments of the present invention is in a pharmaceutically acceptable form. Examples of DHEA congeners include, but are not limited to, compounds having the general formula I, and their metabolites and pharmaceutically acceptable salts thereof: 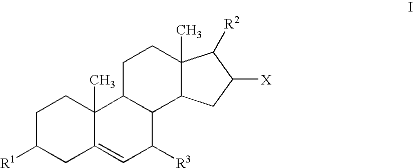
wherein

X is H or halogen;

R¹, R² and R³ are independently ═O, —OH, —SH, H, halogen, pharmaceutically acceptable esters, pharmaceutically acceptable thioesters, pharmaceutically acceptable ethers, pharmaceutically acceptable thioethers, pharmaceutically acceptable inorganic esters, pharmaceutically acceptable monosaccharides, disaccharides or oligosaccharides, spirooxiranes, spirothiranes, —OSO₂R⁴ or —OPOR⁴R⁵;

R⁴ and R⁵ are independently —OH, pharmaceutically acceptable esters or pharmaceutically acceptable ethers.

Suitable metabolites of DHEA include, but are not limited to, dehydroepiandrosterone sulfate, 16α-hydroxydehydroepiandrosterone, 16α-hydroxyandrost-4-ene-3,17dione, androst-4-ene-3,17 dione, 7α-hydroxyandrostenedione, 7α-hydroxytestosterone.

Further examples of DHEA congeners, include but are not limited to, compounds having the general formulas II and III, and their metabolites and pharmaceutically acceptable salts thereof: 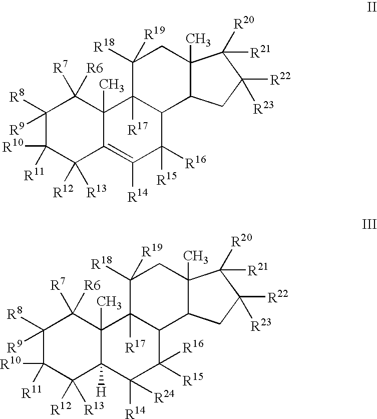
wherein

R⁶, R⁷, R⁸, R⁹, R¹¹, R¹², R¹³, R¹⁴, R¹⁵, R¹⁶, R¹⁷, R¹⁸, R¹⁹ and R²⁴ are independently H, —OH, halogen, C₁₋₁₀ alkyl or C₁₋₁₀ alkoxy;

R¹⁰ is H, —OH, halogen, C₁₋₁₀ alkyl, or C₁₋₁₀ alkoxy;

R²⁰ is (1) H, halogen, C₁₋₁₀ alkyl or C₁₋₁₀ alkoxy when R²¹ is —C(O)OR²⁵ or

(2) H, halogen, OH or C₁₋₁₀ alkyl when R²¹ is H, halogen, OH or C₁₋₁₀alkyl or

(3) H, halogen, C₁₋₁₀ alkyl, C₁₋₁₀ alkenyl, C₁₋₁₀ alkynyl, formyl, C₁₋₁₀ alkanoyl or epoxy when R²¹ is OH; or

R²⁰ and R²¹ taken together are ═O;

R²² and R²³ are independently (1) H, —OH, halogen, C₁₋₁₀ alkyl or C₁₋₁₀ alkoxy when R²¹ is H, OH, halogen, C₁₋₁₀ alkyl or —C(O)OR²⁵ or (2) H, (C₁₋₁₀ alkyl)ₙamino, (C₁₋₁₀ alkyl)ₙamino-C₁₋₁₀ alkyl, C₁₋₁₀ alkoxy, hydroxy-C₁₋₁₀ alkyl, C₁₋₁₀ alkoxy-C₁₋₁₀alkyl, (halogen)ₘ-C₁₋₁₀alkyl, C₁₋₁₀alkanoyl, formyl, C₁₋₁₀ carbalkoxy or C₁₋₁₀ alkanoyloxy when R²⁰ and R²¹ taken together are ═O; or

R²² and R²³ taken together are ═O or taken together with the carbon to which they are attached form a 3-6 member ring containing 0 or 1 oxygen atom; or

R²⁰ and R²² taken together with the carbons to which they are attached form an epoxide ring;

R²⁵ is H, (halogen)ₘ-C₁₋₁₀ alkyl or C₁₋₁₀ alkyl;

n is 0, 1 or 2;

m is 1, 2 or 3; and

physiologically acceptable salts thereof,

with the provisos that

(a) R¹⁰ is not H, halogen, or C₁₋₁₀ alkoxy when R⁶, R⁷, R⁸, R⁹, R¹¹, R¹², R¹³, R¹⁴, R¹⁵, R¹⁷, R¹⁸, R¹⁹ and R²² are H and R¹⁶ is H, halogen, OH or C₁₋₁₀ alkoxy and R²³ is H or halogen and R²⁰ and R²¹ taken together are ═O; and

(b) R¹⁰ is not H, halogen, or C₁₋₁₀ alkoxy when R⁶, R⁷, R⁸, R⁹, R¹¹, R¹², R¹³, R¹⁴, R¹⁵, R¹⁷, R¹⁸, R¹⁹ and R²² are H and R¹⁶ is H, halogen, OH or C₁₋₁₀ alkoxy and R²³ is H or halogen and R²⁰ is H and R²¹ is H, OH or halogen.

The compounds represented by the general formula I exist in many stereoisomers and the formula is intended to encompass the various stereoisomers. Examples of suitable DHEA congeners of Formula I include compounds in which:

(1) R² is ═O, R³ and X are each H and R¹ is ═O, —OH, pharmaceutically acceptable esters thereof, pharmaceutically acceptable ethers thereof or pharmaceutically acceptable salts;

(2) R² is ═O, R³ is H, X is halogen and R¹ is ═O, —OH, pharmaceutically acceptable esters thereof, pharmaceutically acceptable ethers thereof or pharmaceutically acceptable salts;

(3) R² is ═O, R³ and X are each H and R¹ is —SH, pharmaceutically acceptable thioesters thereof, pharmaceutically acceptable thioethers thereof or pharmaceutically acceptable salts;

(4) R² is ═O, R³ is H, X is halogen and R¹ is —SH, pharmaceutically acceptable thioesters thereof, pharmaceutically acceptable thioethers thereof or pharmaceutically acceptable salts;

(5) R² is ═O, X is H and R¹ and R³ are independently ═O, —OH, pharmaceutically acceptable esters thereof, pharmaceutically acceptable ethers thereof or pharmaceutically acceptable salts;

(6) R² is ═O, X is halogen and R¹ and R³ are independently ═O, —OH, pharmaceutically acceptable esters thereof, pharmaceutically acceptable ethers thereof or pharmaceutically acceptable salts;

(7) R² is ═O, X is H and R¹ and R³ are independently —SH, pharmaceutically acceptable thioesters thereof, pharmaceutically acceptable thioethers thereof or pharmaceutically acceptable salts;

(8) R² is ═O, X is halogen and R¹ and R³ are independently —SH, pharmaceutically acceptable thioesters thereof, pharmaceutically acceptable thioethers thereof or pharmaceutically acceptable salts;

(9) R² is —OH, R³ and X are each H and R¹ is ═O, —OH, pharmaceutically acceptable esters thereof, pharmaceutically acceptable ethers thereof or pharmaceutically acceptable salts;

(10) R²is —OH, R³ is H, X is halogen and R¹ is ═O, —OH, pharmaceutically acceptable esters thereof, pharmaceutically acceptable ethers thereof or pharmaceutically acceptable salts;

(11) R² is —OH, R³ and X are each H and R¹ is —SH, pharmaceutically acceptable thioesters thereof, pharmaceutically acceptable thioethers thereof or pharmaceutically acceptable salts;

(12) R² is —OH, R³ is H, X is halogen and R¹ is —SH, pharmaceutically acceptable thioesters thereof, pharmaceutically acceptable thioethers thereof or pharmaceutically acceptable salts;

(13) R²is —OH, X is H and R¹ and R³ are independently ═O, —OH, pharmaceutically acceptable esters thereof, pharmaceutically acceptable ethers thereof or pharmaceutically acceptable salts;

(14) R²is —OH, X is halogen and R¹ and R³ are independently ═O, —OH, pharmaceutically acceptable esters thereof, pharmaceutically acceptable ethers thereof or pharmaceutically acceptable salts;

(15) R² is —OH, X is H and R¹ and R³ are independently —SH, pharmaceutically acceptable thioesters thereof, pharmaceutically acceptable thioethers thereof or pharmaceutically acceptable salts;

(16) R² is —OH, X is halogen and R¹ and R³ are independently —SH, pharmaceutically acceptable thioesters thereof, pharmaceutically acceptable thioethers thereof or pharmaceutically acceptable salts;

(17) R²is —SH, R³ and X are each H and R¹ is ═O, —OH, pharmaceutically acceptable esters thereof, pharmaceutically acceptable ethers thereof or pharmaceutically acceptable salts;

(18) R² is —SH, R³ is H, X is halogen and R¹ is ═O, —OH, pharmaceutically acceptable esters thereof, pharmaceutically acceptable ethers thereof or pharmaceutically acceptable salts;

(19) R² is —SH, R³ and X are each H and R¹ is —SH, pharmaceutically acceptable thioesters thereof, pharmaceutically acceptable thioethers thereof or pharmaceutically acceptable salts;

(20) R² is —SH, R³ is H, X is halogen and R¹ is —SH, pharmaceutically acceptable thioesters thereof, pharmaceutically acceptable thioethers thereof or pharmaceutically acceptable salts;

(21) R² is —SH, X is H and R¹ and R³ are independently ═O, —OH, pharmaceutically acceptable esters thereof, pharmaceutically acceptable ethers thereof or pharmaceutically acceptable salts;

(22) R² is —SH, X is halogen and R¹ and R³ are independently ═O, —OH, pharmaceutically acceptable esters thereof, pharmaceutically acceptable ethers thereof or pharmaceutically acceptable salts;

(23) R² is —SH, X is H and R¹ and R³ are independently —SH, pharmaceutically acceptable thioesters thereof, pharmaceutically acceptable thioethers thereof or pharmaceutically acceptable salts;

(24) R² is —SH, X is halogen and R¹ and R³ are independently —SH, pharmaceutically acceptable thioesters thereof, pharmaceutically acceptable thioethers thereof or pharmaceutically acceptable salts;

(25) X is H and R¹ is ═O, —OH, pharmaceutically acceptable esters thereof, pharmaceutically acceptable ethers thereof or pharmaceutically acceptable salts, R² and R³ are independently ═O, —OH, a sugar residue, pharmaceutically acceptable esters thereof, pharmaceutically acceptable ethers thereof or pharmaceutically acceptable salts, wherein at least one of R² and R³ is a sugar residue;

(26) X is halogen and R¹ is ═O, —OH, pharmaceutically acceptable esters thereof, pharmaceutically acceptable ethers thereof or pharmaceutically acceptable salts, R² and R³ are independently ═O, —OH, a sugar residue, pharmaceutically acceptable esters thereof, pharmaceutically acceptable ethers thereof or pharmaceutically acceptable salts, wherein at least one of R² and R³ is a sugar residue;

(27) X is H, R¹ is ═O or —OH, and R² and R³ are independently ═O, —OH, pharmaceutically acceptable inorganic esters thereof or pharmaceutically acceptable salts, wherein at least one of R² and R³ is an inorganic ester; and/or

(28) X is halogen R¹ is ═O or —OH, and R² and R³ are independently ═O, —OH, pharmaceutically acceptable inorganic esters thereof or pharmaceutically acceptable salts, wherein at least one of R² and R³ is an inorganic ester.

Pharmaceutically acceptable esters or thioesters include, but are not limited to, esters or thioesters of the formula —OOCR or —SOCR, wherein R is a pharmaceutically acceptable alkyl, alkenyl, aryl, alkylaryl, arylalkyl, sphingosine or substituted sphingolipid groups, such as propionate, enanthate, cypionate, succinate, decanoate and phenylpropionate esters.

Pharmaceutically acceptable ethers or thioethers include, but are not limited to, ethers or thioethers of the formula —OR or —SR, wherein R is as defined above or enol, or —OR is an unsubstituted or substituted spirooxirane or —SR is a spirothiane.

Suitable sugar residues can include, but are not limited to monosaccharides, disaccharides, and oligosaccharides, such as a glucuronate.

Pharmaceutically acceptable inorganic esters include, but are not limited to, esters of the formula —OSO₂R⁴ or —OPOR⁴R⁵, wherein R⁴ and R⁵ are independently —OH, pharmaceutically acceptable esters, pharmaceutically acceptable ethers or pharmaceutically acceptable salts.

Some DHEA congeners, such as the compounds of general formulas I, II, and III, can be synthesized as described in U.S. Pat. Nos. 4,898,694; 5,001,119; 5,028,631; and 5,175,154, which are all incorporated herein by reference. The compounds represented by the general formulas II and III exist in many stereoisomers and these formulas are intended to encompass the various stereoisomers. Examples of representative compounds, which fall within the scope of general formulas II and III, include the following: 5α-androstan-17-one; 16α-fluoro-5α-androstan-17-one; 3β-methyl-5α-androsten-17-one; 16β-fluoro-5α-androstan-17-one; 17β-bromo-5-androsten-16-one; 17β-fluoro-3β-methyl-5-androsten-16-one; 17α-fluoro-5α-androstan-16-one; 3β-hydroxy-5-androsten-17-one; 17α-methyl-5α-androstan-16-one; 16α-methyl-5-androsten-17-one; 3β,16α-dimethyl-5-androsten-17-one; 3β,17α-dimethyl-5-androsten-16-one; 16α-hydroxy-5-androsten-17-one; 16α-fluoro-16β-methyl-5-androsten-17-one; 16α-methyl-5α-androstan-17-one; 16-dimethylaminomethyl-5α-androstan-17-one; 16β-methoxy-5-androsten-17-one; 16α-fluoromethyl-5-androsten-17-one; 16-methylene-5-androsten-17-one; 16-cyclopropyl-5α-androstan-17-one; 16-cyclobutyl-5-androsten-17-one; 16-hydroxymethylene-5-androsten-17-one; 3α-bromo-16α-methoxy-5-androsten-17-one; 16-oxymethylene-5-androsten-17-one; 3β-methyl-16ε-trifluoromethyl-5α-androstan-17-one; 16-carbomethoxy-5-androsten-17-one; 3β-methyl-16β-methoxy-5α-androstan-17-one; 3β-hydroxy-16α-dimethylamino-5-androsten-17-one; 17α-methyl-5-androsten-17β-ol; 17α-ethynyl-5α-androstan-17β-ol; 17β-formyl-5α-androstan-17β-ol; 20,21 -epoxy-5α-pregnan-17α-ol; 3β-hydroxy-20,21-epoxy-5α-pregnan-17α-ol; 16α-fluoro-17α-ethenyl-5-androsten-17β-ol; 16α-hydroxy-5-androsten-17α-ol; 16α-methyl-5α-androstan-17α-ol; 16α-methyl-16β-fluoro-5α-androstan-17α-ol; 16α-methyl-16β-fluoro-3-hydroxy-5-androsten-17α-ol; 3β,16β-dimethyl-5-androsten-17β-ol; 3β,16,16-trimethyl-5-androsten-17β-ol; 3β,16,16-trimethyl-5-androsten-17-one; 3β-hydroxy-4α-methyl-5-androsten-17α-ol; 3β-hydroxy-4α-methyl-5-androsten-17-one; 3α-hydroxy-1α-methyl-5-androsten-17-one; 3α-ethoxy-5α-androstan-17β-ol; 5α-pregnan-20-one; 3β-methyl-5α-pregnan-20-one; 16α-methyl-5-pregnen-20-one; 16α-methyl-3β-hydroxy-5-pregnen-20-one; 17α-fluoro-5-pregnen-20-one; 21-fluoro-5α-pregnan-20-one; 17α-methyl-5-pregnen-20-one; 20-acetoxy-cis-17(20)-5α-pregnene; and 3α-methyl-16,17-epoxy-5-pregnen-20-one, for example.

In one aspect of the present invention, a DHEA congener and a second anti-inflammatory agent can be co-administered to a subject in an amount that results in a therapeutic effect, thereby aiding in treating and/or preventing inflammation in a subject. The dose of the DHEA congener administered is selected to achieve DHEA or DHEA equivalent blood levels greater than normal endogenous DHEA blood levels. Normal endogenous blood levels of DHEA can be less than 20 ng/mL. Accordingly, peak blood levels of DHEA or DHEA equivalent can be greater than about 20 ng/mL, or as desired for a specific therapeutic effect. In one aspect, suitable doses that are selected to achieve a peak blood level of DHEA or DHEA equivalent can be in the range from about 20 ng/mL to about 100 mg/mL, or in the range from about 30 ng/mL to about 10 mg/mL. Additionally, the doses administered to a subject can be in an amount to achieve DHEA blood levels in the subject from about 100 ng/mL to about 1 mg/mL, from 100 ng/mL to about 100 μg/mL, and/or from about 100 ng/mL to about 10 μg/mL.

In accordance with the methods of the present invention, a DHEA congener can be administered as a part of a regimen to aid in the reduction of subchronic to chronic inflammation as well as acute inflammation. In one aspect, a DHEA congener can be administered in a dosing regimen that includes providing from about 1 mg to about 200 mg per day of the DHEA congener to a subject to aid in reducing subchronic to chronic inflammation. In another aspect, a DHEA congener can be administered in a dosing regimen that includes providing from about 10 mg to about 3600 mg of the DHEA congener to a subject to aid in reducing acute inflammation. In still a further aspect of the present invention, a DHEA congener can be administered in a dosing regimen to aid in preventing the onset of inflammation, which includes providing from about 10 mg to about 3600 mg per day of the DHEA congener to a subject not yet experiencing observable inflammation. These dosages can be administered once a day, or at smaller dosages throughout the day.

B) Parthenolide

Parthenolide is a chemical found in the feverfew plant (Chrysanthemum parthenium, also known as Tanacetum parthenium and bachelor's button) and has the following structure: 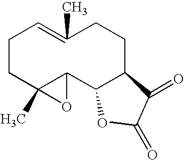
Parthenolide is also member of a large and diverse group of biologically active plant chemicals called sesquiterpene lactones. Parthenolide has a number of biological effects, including anti-inflammatory, anti-secretory, and spasmolytic activity, as well as inhibition of activation of MAP kinase. Additionally, parthenolide may be beneficial in the treatment of cancer, e.g., it exerts anti-proliferative effects on certain cancer cell lines and induces apoptosis in leukemia cells.

Parthenolide is typically most concentrated in the leaves and flowers of feverfew plants. Preparations made both from fresh and from dried feverfew plants are effective in the treatment of arthritis and migraines and can be orally administered as a dried powder, or in the form of capsules, tablets, and liquid extracts. Exemplary preparations can contain from about 0.2% to about 0.7% parthenolide.

Parthenolide may be extracted from feverfew plants by extraction methods that are well-known in the art. These include extraction using liquid solvents such as hexanes, chloroform, ethanol, water, or a mixture of such solvents. Such methods commonly involve steeping the plant matter in the solvent to allow the soluble materials to dissolve, then filtering the resulting liquid to obtain an extract of parthenolide. This product may then be vacuum-filtered and freeze-dried to yield a solid. Another extraction technique could involve passing supercritical carbon dioxide gas through feverfew plant material, where the gas serves as a solvent. This process usually yields a thick resin or oil that contains less solvent residue than with other techniques, lessening the need for further solvent-removal steps.

Plant-based active ingredients are often present in their host plants in low concentrations. Accordingly, extracts of these plants may also contain relatively low concentrations of these ingredients. However, dosing and administration of therapeutically effective amounts of these ingredients to a subject may benefit from using a purer form. The extraction products of such techniques as described above may be fractionated using chromatographic techniques to yield a purer form of parthenolide. Use of pure parthenolide may make it possible to deliver a therapeutically effective amount to a subject in fewer or smaller doses. It should be noted that the present invention is not directed at methods of producing pure parthenolide or parthenolide extracts. However, both extracted and pure parthenolide produced by the above techniques or other techniques known in the art may be used in accordance with the present invention.

In accordance with the methods of the present invention, a parthenolide may be administered as part of a regimen to aid in the reduction of subchronic to chronic inflammation as well as acute inflammation. The parthenolide may be either from an extract or it may be a purified form. In addition, the parthenolide may be in the form of a parthenolide derivative, or it may be a non-derivatized form. In one aspect of the present invention, the parthenolide may be administered in a dosing regimen that includes providing from about 0.01 mg to about 1000 mg per day of the parthenolide to a subject to aid in reducing inflammation. In another aspect, the parthenolide may be administered in an amount from 0.1 mg to 500 mg per day. As with DHEA, administration of larger amounts of parthenolide may be indicated for reducing acute inflammation or for preventing inflammation than are indicated for reducing subchronic or chronic inflammation.

The present invention is related to co-administration of DHEA congeners with a parthenolide. In the present invention, a parthenolide and a DHEA congener may be co-administered to a subject to reduce inflammation. In another aspect of the invention, a DHEA congener and a parthenolide may be co-administered preemptively to a subject not yet exhibiting symptoms of inflammation, in order to inhibit inflammation. This co-administration can result in an enhancing effect, or even a synergistic effect. Therefore, co-administration of the DHEA congener and the parthenolide may result in a greater anti-inflammatory response than administration of either the DHEA congener or the parthenolide alone. In an alternative embodiment, co-administration of the DHEA congener and the parthenolide may result in an anti-inflammatory response that is greater than the sum of the responses to independent administration of the DHEA congener and the parthenolide at their respective doses.

As such, DHEA can be co-administered with a parthenolide in accordance with methods of the present invention to more effectively reduce inflammation or treat inflammatory producing diseases. Examples of conditions that would benefit from the methods of the present invention include rheumatoid arthritis, asthma, ulcerative colitis, multiple sclerosis, chronic obstructive pulmonary disease (COPD), allergic rhinitis, rheumatic fever, thrombocytopenia, kidney inflammation, lupus, acrotic dermatis, tissue necrosis, tuberculosis, chronic cholecystitis, bronchiectasis, Hashimoto's thyroiditis, pneumoconiosis, pelvic inflammatory disease, pancreatitis, cardiovascular disease, psoriatic arthritis, psoriasis, sarcoidosis, Adult Still's disease, spondyloarthropathies, ankylosing spondylitis, Bechet's syndrome, Crohn's disease, orofacial Crohn's disease, uveitis, graft-vs-host disease, advanced heart failure, common variable immunodeficiency, Wegener's granulomatosis, sepsis, pyoderma gangrenosum, subcorneal pustular dermatosis, Hidradenitis suppurativa, panniculitis, and Langerhans' cell histiocytsis, as well as other maladies that end with the suffix “itis.”

Administration of the DHEA congener as well as the parthenolide may be accomplished by a number of routes, including oral, nasal, transdermal, parenteral, intravenous, intraarterial, intramuscular, and subcutaneous. Co-administration of these agents in accordance with embodiments of the present invention may involve using a common administration route for both. Alternatively, each may be administered independently by a separate route and still constitute co-administration in accordance with the present invention, so long as the agents work in concert to produce a physiological effect.

Use of a DHEA congener and a parthenolide to treat any of these or other inflammatory conditions will involve accounting for administration considerations that are specific to each disease, such as specific dosage amounts, dosage timing, dosage periods, administration routes, etc. These and other administration considerations can be varied by one skilled in the art (for administration with DHEA congener) in order to achieve a therapeutic effect. In other words, as one skilled in the art understands, dosages, timing of administration, length of administration, administration routes, drug selection, etc., should considered on a case by case basis.

C) Preparation and Dosage Forms

The present invention also provides for compositions for reducing inflammation in a subject. Such a composition may comprise a DHEA congener, a parthenolide, and a carrier. The DHEA congener can be present in any form described herein, or other suitable derivatized forms. The parthenolide may be either from an extract or it may be a purified form. In addition, the parthenolide may be in the form of a parthenolide derivative, or it may be a non-derivatized form. The amounts of each of the DHEA congener and the parthenolide can be any pharmaceutically effective amount to achieve a therapeutic effect. As a guideline, the amounts set forth herein are exemplary of suitable dosages.

Pharmaceutical compositions containing compounds of the present invention as the active ingredient can be prepared according to conventional pharmaceutical compounding techniques known to one of ordinary skill in the art. Typically, a therapeutically effective amount of the active ingredient can be admixed with a pharmaceutically acceptable carrier. The carrier may take a wide variety of forms depending on the desired route of administration, e.g., oral, parenteral, intravenous, intraarterial, intrathecal epidural, intramuscular, transdermal, subcutaneous, transbuccal, ocular, nasal, or suppository. The compositions may further contain antioxidizing agents, stabilizing agents, preservatives, or the like. Broader categories of dosage forms include oral dosage forms, parenteral dosage forms, and transdermal/mucosal dosage forms.

Liquid carriers can be used in preparing solutions, suspensions, emulsions, syrups, elixirs and pressurized compositions. The active ingredients can be dissolved or suspended in a pharmaceutically acceptable liquid carrier such as water, cyclodextrins, an organic solvent, pharmaceutically acceptable oils, and/or fats. The liquid carrier can contain other suitable pharmaceutical additives such as solubilizers, emulsifiers, buffers, preservatives, sweeteners, flavoring agents, suspending agents, thickening agents, coloring agents, viscosity regulators, stabilizers and/or osmo-regulators. Suitable examples of liquid carriers for oral administration can include water, alcohols (including monohydric alcohols and polyhydric alcohols, e.g., glycols) and their derivatives, oils (e.g., peanut oil, sesame oil, olive oil, and coconut oil), and combinations of the above. Compositions comprising such carriers and adjuvants may be formulated using well-known conventional materials and methods.

A solid carrier can be formulated into capsules, pills, tablets, lozenges, melts, or powders. A solid carrier can include starches, sugars, bicarbonates, diluents, granulating agents, disintegrating, and/or dispersing agents. The formulations can include one or more substance(s) which may also act as flavoring agents, lubricants, solubilizers, suspending agents, fillers, glidants, compression aids, binders, or tablet-disintegrating agents, for example. In powders, the carrier can be a finely divided solid which is in an admixture with the finely divided active ingredients. The carrier and drug can form a single composite with drug adsorbed to its surface that effectively enhances the rate of dissolution in the gastrointestinal tract. The powders and/or tablet can contain up to 100 wt % of the active ingredients, though typically this will not be the case, and can be formulated for immediate and/or sustained release of the active ingredient.

With respect to tablets, the active ingredients can be mixed with a carrier having the necessary compression properties in suitable proportions and compacted in the shape and size desired. Exemplary forms can include dry powder compaction tablets, micro-particulate systems, e.g., wherein the active ingredient is spray-dried onto a scaffold particle, and hard or soft-gel capsules. In one embodiment, the tablets can be optionally covered with an enteric coating, which remains intact in the stomach, but will dissolve and release the contents of the tablet once it reaches the small intestine. Alternatively, the tablets can be formulated to enhance gastric uptake to avoid first pass effect in the liver following intestinal absorption.

The composition can include one or more sustained or controlled release excipient(s) such that a slow, sustained, or constant release of the active ingredients can be achieved. A wide variety of suitable excipients are known in the art. Such sustained/controlled release excipients and systems are described, for example, in U.S. Pat. Nos. 5,612,053; 5,554,387; 5,512,297; 5,478,574; and 5,472,711, each of which is incorporated by reference herein. If desired, the pharmaceutical composition can be formulated to provide a pulse dose of the active ingredient. A variety of pulse-dose systems, which provide low or high-pulsed doses, are known in the art. In another embodiment of the invention, the pharmaceutical can be formulated to provide direct and/or targeted delivery of the active ingredient to a specific anatomic site or sites within the gastrointestinal tract; e.g., the duodenum, jejunum, ileum, cecum and/or colon. Methods for providing targeted delivery of pharmaceuticals to specific tissues or organs within a mammalian host are well known in the art.

To achieve a therapeutic level in systemic circulation, a compound, e.g., including active agent(s), can be formulated using standard techniques to form a composition having a high bioavailability of the active agents in order to meet the desired therapeutic blood levels. For example, in one embodiment, the active agent(s), a complex of one or more of the active agent(s) and cyclodextrin, or the active agent(s) in a nanoparticle delivery system, may be dissolved or suspended in a pharmaceutical carrier and administered as either a solution or a suspension. Cyclodextrins of all classes (alpha, beta and gamma) and their substituted or derivatized forms can be used, as well as mixtures thereof. In one aspect of the present invention, a complex of the active agent(s) with a cyclodextrin or the active agent in a nanoparticle delivery system can be used. In another aspect, a complex of the active agent(s) and a cyclodextrin, such as a 2-hydroxypropyl β-cyclodextrin, can be prepared in accordance with U.S. Pat. No. 4,727,064 and/or European Patent No. 0 149 197, each of which are incorporated herein by reference. The use of the compound as part of a cyclodextrin complex or nanoparticle delivery system can allow for the preparation of both parenteral and oral solutions and oral solid dosage forms with high concentration of active agent. Illustrative of suitable carriers include water, saline, dextrose solutions, fructose solutions, ethanol, or oils of animal, vegetative or synthetic origin. The carrier may also contain other ingredients including, for example, preservatives, suspending agents, solubilizing agents, buffers, and/or the like. When the compounds are being administered intrathecally, they or their cyclodextrin complexes or nanoparticle delivery systems may also be dissolved in cerebrospinal fluid.

The active agent(s) can be administered in a therapeutically effective amount. The actual amount administered, and the rate and time-course of administration, can depend on the nature and severity of the condition being treated. Thus, it may be desirable to administer the highly bioavailable complex of the active agent(s) at several intervals during a dosing regimen to maintain blood levels at the therapeutic index. For example, depending on the dosing regimen, it can be preferable to administer the formulated active agent(s) two to four times per day, and more preferably to administer it two to three times per day. Alternatively, one might use an oral controlled release method to meter the drug available for absorption over a 24 hour period, thereby necessitating only a single dose per day. Prescription of treatment, e.g. decisions on dosage, timing, periods of administration, drug selection, etc., can be within the responsibility of general practitioners or specialists, and typically can take into account the disorder to be treated, the condition of the individual patient, the site of delivery, the method of administration, and other factors known to practitioners.

As will be appreciated by those of skill in the art, the form of the pharmaceutical composition of the active agent(s) and the mode of administration will determine the dose of the active agent(s) to be delivered. A factor to consider in determining the proper dose to meet the desired peak blood levels is the bioavailability of the active agent(s) in the pharmaceutical composition, i.e., the availability of the active agent(s) for raising blood levels of DHEA or DHEA equivalent. For example, the bioavailability of the active agent(s) in a pharmaceutical composition delivered intravenously can be greater than that for the same pharmaceutical composition delivered orally. Thus, a lower dose of a pharmaceutical composition containing the active agent(s) can be administered intravenously than that which would be used orally. For oral administration of active agent(s) with low water solubility, it is well recognized that co-formulation of the agent(s) with a substance that accelerates dissolution or enhances solubility can increase the active agent's bioavailability. For example, active agent used to increase blood levels of DHEA congener or equivalent can be many times more soluble in water if complexed with a cyclodextrin than without. Similarly, the same active agent can dissolve in water much faster and have higher bioavailability if adsorbed to a high surface area particle with a large surface area. For example, suitable blood levels of the DHEA congener can be achieved by the administration of 100 mg of a cyclodextrin-DHEA complex intravenously, 600 mg of a cyclodextrin-DHEA complex orally, or 500 mg of DHEA in a nanoparticle delivery system orally. As will also be appreciated by those of skill in the art, the form of the pharmaceutical composition of the DHEA congener and the parthenolide can depend on the intended mode of administration, which in turn will depend on the location and nature of the disorder to be treated. Accordingly, delivery to the gastrointestinal tract, e.g., for treatment of gastrointestinal mucositis, peptic ulcers and inflammatory bowel diseases, such as Crohn's disease, ulcerative colitis, indeterminate colitis, and infectious colitis, can be in the form of oral solutions, gels, suspensions, tablets, capsules, and the like.

The following example illustrates the embodiment of the invention that is presently best known. However, it is to be understood that the following is only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following example provides further detail in connection with what are presently deemed to be the most practical and preferred embodiments of the invention.

### EXAMPLE

### Synergistic Effect of DHEA and Parthenolide on TNF-α Release

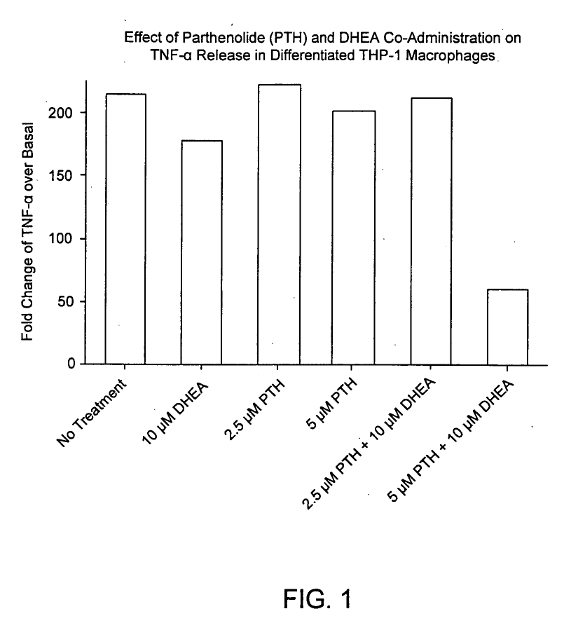

THP-1 macrophages were stimulated with lipopolysaccharide and treated with either DHEA, parthenolide (PTH), or a combination of the two compounds. The amount of TNF-α released was measured. The fold change in TNF-α over basal levels as a result of each treatment is shown in FIG. 1. Treatment with a combination of 5 μM parthenolide and 10 μM DHEA produced a greater suppression of TNF-α than treatment with either 5 μM parthenolide or 10 μM DHEA alone. This suppression was also greater than the sum of the suppressive effects produced from treatment with 5 μM parthenolide and 10 μM DHEA.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A method of reducing inflammation in a subject, comprising co-administering therapeutically effective amounts of a DHEA congener and a parthenolide to the subject.

2. A method as in claim 1, wherein the parthenolide is non-derivatized.

3. A method as in claim 1, wherein reducing inflammation includes preemptively co-administering the DHEA congener and the parthenolide to the subject to inhibit inflammation.

4. A method as in claim 1, wherein reducing inflammation includes treating the subject experiencing inflammation.

5. A method as in claim 1, wherein the co-administering step results in enhanced anti-inflammatory response compared to the administration of either the DHEA congener or the parthenolide alone at their respective dosages.

6. A method as in claim 1, wherein the DHEA congener and parthenolide are each administered at a therapeutically effective dosage, such that the inflammation is reduced more than by the summation of inflammation reduction for each administered alone.

7. A method as in claim 1, wherein reducing inflammation includes treating a disease selected from the group of diseases consisting of: rheumatoid arthritis, asthma, ulcerative colitis, multiple sclerosis, chronic obstructive pulmonary disease (COPD), allergic rhinitis, rheumatic fever, thrombocytopenia, kidney inflammation, lupus, acrotic dermatis, tissue necrosis, tuberculosis, chronic cholecystitis, bronchiectasis, Hashimoto's thyroiditis, pneumoconiosis, pelvic inflammatory disease, pancreatitis, cardiovascular disease, psoriatic arthritis, psoriasis, sarcoidosis, Adult Still's disease, spondyloarthropathies, ankylosing spondylitis, Bechet's syndrome, Crohn's disease, orofacial Crohn's disease, uveitis, graft-vs-host disease, advanced heart failure, common variable immunodeficiency, Wegener's granulomatosis, sepsis, pyoderma gangrenosum, subcorneal pustular dermatosis, Hidradenitis suppurativa, panniculitis, and Langerhans' cell histiocytsis.

8. A method as in claim 1, wherein the DHEA congener is administered to the subject in an amount from 10 mg to 3600 mg per day.

9. A method as in claim 8, wherein the DHEA congener is administered to the subject in an amount from 1 mg to 200 mg per day.

10. A method as in claim 1, wherein a serum level from 20 ng/ml to 100 mg/ml of DHEA is achieved in the subject.

11. A method as in claim 10, wherein a serum level from 10 ng/ml to 30 ng/ml of DHEA is achieved in the subject.

12. A method as in claim 1, wherein the parthenolide is administered to the subject in an amount from 0.001 mg to 1000 mg per day.

13. A method as in claim 12, wherein the parthenolide is administered to the subject in an amount from 0.1 mg to 500 mg per day.

14. A method as in claim 1, wherein the DHEA congener and the parthenolide are each administered to the subject by a route selected from the group consisting of oral, nasal, transdermal, parenteral, intravenous, intraarterial, intramuscular, and subcutaneous.

15. A method as in claim 14, wherein the DHEA congener and the parthenolide are both administered to the subject by the same route

16. A method as in claim 15, wherein the DHEA congener and the parthenolide are both administered to the subject orally.

17. A method as in claim 1, wherein the subject is human.

18. A composition for reducing inflammation in a subject, comprising:
a DHEA congener;
a parthenolide; and
a carrier,
said composition effective for enhancing an anti-inflammatory response in the subject compared to the administration of either the DHEA congener or the parthenolide alone at their respective dosages.

19. A composition as in claim 18, wherein the anti-inflammatory response is an inflammation preventative response resulting from preemptively co-administering the DHEA congener and the parthenolide to the subject to inhibit inflammation.

20. A composition as in claim 18, wherein the anti-inflammatory response is an inflammation reducing response resulting from co-administering the DHEA congener and the parthenolide to the subject experiencing inflammation.

21. A composition as in claim 18, wherein the parthenolide is non-derivatized.

22. A composition as in claim 18, wherein the composition is in an oral dosage form.

23. A composition as in claim 22, wherein the oral dosage form is a liquid solution or suspension.

24. A composition as in claim 22, wherein the oral dosage form is a solid.

25. A composition as in claim 18, wherein the composition is in a parenteral dosage form.

26. A composition as in claim 18, wherein the composition is in a dosage form selected from the group consisting of transmucosal and transdermal.

27. A composition as in claim 18, wherein the composition is in a sustained-release form.

28. A composition as in claim 18, wherein at least one of DHEA congener or the parthenolide is complexed to a cyclodextrin.

29. A composition as in claim 18, wherein the composition is in the form of nanoparticles.

30. A composition as in claim 18, wherein the subject is human.
